(19) United States
(12) Patent Application Publication
(10) Pub. No.: US 2016/0361555 A1
(43) Pub. Date: Dec. 15, 2016

(54) MULTIVECTOR PATIENT ELECTRODE SYSTEM AND METHOD OF USE

(71) Applicants: Walter T. Savage M.D., Concord, CA (US); Shelley J. Savage, Concord, CA (US); Douglas M. Raymond, Livermore, CA (US); Peter D. Gray, Vallejo, CA (US); Paul A. Friedman M.D., Rochester, MN (US); Samuel J. Asirvatham M.D., Rochester, MN (US); Charles J. Bruce M.D., Rochester, MN (US); Christopher V. DeSimone M.D., Rochester, MN (US)

(72) Inventors: Walter T. Savage M.D., Concord, CA (US); Shelley J. Savage, Concord, CA (US); Douglas M. Raymond, Livermore, CA (US); Peter D. Gray, Vallejo, CA (US); Paul A. Friedman M.D., Rochester, MN (US); Samuel J. Asirvatham M.D., Rochester, MN (US); Charles J. Bruce M.D., Rochester, MN (US); Christopher V. DeSimone M.D., Rochester, MN (US)

(21) Appl. No.: 14/736,188

(22) Filed: Jun. 10, 2015

(51) Int. Cl. A61N 1/39; A61N 1/05

(52) U.S. Cl. CPC A61N 1/3956; A61N 1/0563; A61N 1/3918; A61N 1/3925

(57) ABSTRACT

A multi-vector patient electrode system and method of use are disclosed.

### FIELD

The disclosure relates generally to methods and arrangements relating to medical devices. More specifically, the disclosure relates to systems and methods used in medical device patient electrode systems especially as used in subcutaneous implantable cardioverter defibrillators, implantable cardioverter defibrillators, substernal implantable defibrillators and epicardial defibrillators.

### BACKGROUND

A primary task of the heart is to pump oxygenated, nutrient-rich blood throughout the body. Electrical impulses generated by a portion of the heart regulate the pumping cycle. When the electrical impulses follow a regular and consistent pattern, the heart functions normally and the pumping of blood is optimized. When the electrical impulses of the heart are disrupted (i.e., cardiac arrhythmia), this pattern of electrical impulses becomes chaotic or overly rapid, and a Sudden Cardiac Arrest may take place, which inhibits the circulation of blood. As a result, the brain and other critical organs are deprived of nutrients and oxygen. A person experiencing Sudden Cardiac Arrest may suddenly lose consciousness and die shortly thereafter if left untreated.

The most successful therapy for Sudden Cardiac Arrest is prompt and appropriate defibrillation. A defibrillator uses electrical shocks to restore the proper functioning of the heart. A crucial component of the success or failure of defibrillation, however, is time. Ideally, a victim should be defibrillated immediately upon suffering a Sudden Cardiac Arrest, as the victim's chances of survival dwindle rapidly for every minute without treatment.

There are a wide variety of defibrillators. A common type of defibrillator is the automated external defibrillator (AED). The AED is an external device used by a third party to resuscitate a person who has suffered from sudden cardiac arrest. FIG. 1 illustrates a conventional AED 100, which includes a base unit 102 and two pads 104. Sometimes paddles with handles are used instead of the pads 104. The pads 104 are connected to the base unit 102 using electrical cables 106.

A typical protocol for using the AED 100 is as follows. Initially, the person who has suffered from sudden cardiac arrest is placed on the floor. Clothing is removed to reveal the person's chest 108. The pads 104 are applied to appropriate locations on the chest 108, as illustrated in FIG. 1. The electrical system within the base unit 100 generates a high voltage between the two pads 104, which delivers an electrical shock to the person. Ideally, the shock restores a normal cardiac rhythm. In some cases, multiple shocks are required.

Although existing technologies work well, there are continuing efforts to improve the effectiveness, safety and usability of automatic external defibrillators. Accordingly, efforts have been made to improve the availability of automated external defibrillators (AED), so that they are more likely to be in the vicinity of sudden cardiac arrest victims. Advances in medical technology have reduced the cost and size of automated external defibrillators (AED). Some modern AEDs approximate the size of a laptop computer or backpack. Even small devices may typically weigh 4-10 pounds or more. Accordingly, they are increasingly found mounted in public facilities (e.g., airports, schools, gyms, etc.) and, more rarely, residences. Unfortunately, the average success rates for cardiac resuscitation remain abysmally low (less than 8.3%).

Another type of defibrillator is the Wearable Cardioverter Defibrillator (WCD). Rather than a device being implanted into a person at-risk from Sudden Cardiac Arrest, or being used by a bystander once a person has already collapsed from experiencing a Sudden Cardiac Arrest, the WCD is an external device worn by an at-risk person which continuously monitors their heart rhythm to identify the occurrence of an arrhythmia, to then correctly identify the type of arrhythmia involved and then to automatically apply the therapeutic action required for the type of arrhythmia identified, whether this be cardioversion or defibrillation. These devices are most frequently used for patients who have been identified as potentially requiring an ICD and to effectively protect them during the two to six month medical evaluation period before a final decision is made and they are officially cleared for, or denied, an ICD.

Manual external defibrillators and WCDs are also used for external cardioversion, which is where a shaped electrical pulse is used to terminate atrial fibrillation in a patient. This also requires the use of external electrode pads.

External Defibrillators and Automated External Defibrillators on the market today make use of either rigid paddles that must be held in place on the patient's body or else flexible electrode pads (made of conductive foil and foam) which are stuck to the patient's skin. The current external defibrillators that have rigid paddle bases do not conform to the curvatures of the patient's body at the locations on the body where the paddles must be placed in order to be effective. As such the operators of these devices must apply a good amount of contact force to make physical contact across the paddle's patient contact interface and must maintain this force to maximize the surface area in contact with the patient for the sensing and reading of the heart rhythm in order that the device can detect the presence of a faulty rhythm, or arrhythmia, such as Ventricular Fibrillation or Ventricular Tachycardia so as to instruct/initiate or signal the external defibrillator to deliver the life saving therapeutic defibrillation shock pulse. The operator must also continue holding the required contact force while the device delivers the chosen therapeutic action (shock or no shock).

Wearable Cardioverter Defibrillators on the market today are still bulky and uncomfortable for the patients to wear. They utilize a single source of energy in a box that attaches to the wearable garment (containing the sensors and the electrodes) and the energy source box normally rides on the hip. These are heavy and uncomfortable to wear and a frequent source of complaints from patients.

Current Wearable Cardioverter Defibrillators have fixed flat surface electrodes and fixed curved surface electrodes for positioning on the patient's back and abdomen. This requires that each patient has to be specially fitted for their own unit, which is time consuming for the patient. Given the limited range of device sizes available it also requires that the device be worn tightly in order to maintain a constant contact pressure with both the sensors and the electrodes, which is restrictive and can be uncomfortable for the patient. This is also the reason why the devices also employ the use of liquid conductive hydrogel, to ensure that the electrode-to-patient contact impedance is minimized. This is messy to clean up after each use when deployed by the device, and naturally this can adversely impact the patient's clothing. It also requires that the liquid reservoirs be recharged before the device can be effectively used again.

For patients who are significantly ill, and who are known to be at an elevated risk of imminent cardiac arrest, Implantable Cardioverter Defibrillators (ICDs) as illustrated in FIG. 2 are prescribed and then surgically implanted into the patient for either primary or secondary prevention purposes. ICDs are fully automated devices which involve wire coils 202, electrical leads 201 and a generator device 200 being implanted within a person, with the coil(s) in direct contact with the cardiac tissue and the transvenous lead(s) 201 connecting back to the generator. When a life threatening cardiac arrhythmia is detected, the appropriate current is then automatically passed through the heart of the user with little or no intervention by a third party.

Subcutaneous Implantable Cardioverter Defibrillators (S-ICDs) as illustrated in FIG. 3 have also recently become available, since they offer all of the advantages of an implantable ICD (rapid defibrillation for high risk individuals), without the long-term risks associated with transvenous leads (lead failure due to repetitive cardiac motion, infection leading to septicemia, lead thrombus and thromboembolism, inappropriate shocks from lead failure). Since S-ICDs do not touch the heart, a greater amount of energy is required for effective defibrillation, leading to larger, bulkier devices and shorter generator longevity. Current systems utilize a left-lateral pulse generator 301 connected to a lead 302 tunneled over the sternum.

One of the major shortcomings of existing dual electrode approaches are that they only enable a single path of the defibrillation shock, known as a shock vector, across the heart. The placement of the electrodes is known to affect the transmyocardial current. Defibrillation success depends on delivering sufficient peak transmyocardial current in order to depolarize a critical myocardial mass (thought to be in the range of 72-80% of ventricular mass). The responsiveness of individual cardiac fibers and myocytes to the electrical pulse is also thought to be linked to the physical alignment, within 3 dimensions, of the cardiac fibers and myocytes compared to the vector of the therapeutic electrical pulse.

### BRIEF DESCRIPTION OF THE DRAWINGS

FIG. 1 diagrammatically illustrates an example of a conventional external defibrillator.

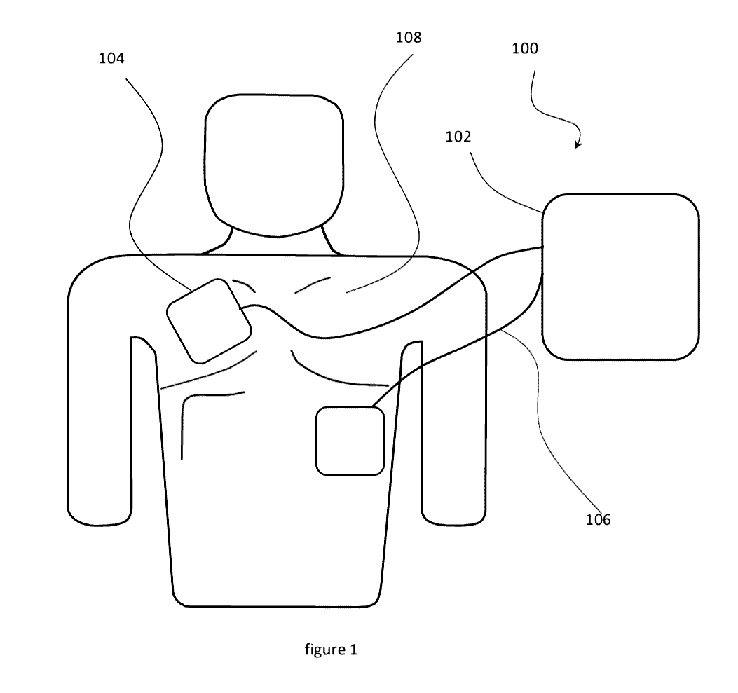

FIG. 2 illustrates an example of a standard implantable cardioverter defibrillator.

FIG. 3 illustrates an example of a standard subcutaneous implantable cardioverter defibrillator.

FIG. 4 illustrates a subcutaneous implantable cardioverter defibrillator with multiple shock electrodes and multiple sensing electrodes.

FIG. 5 illustrates a subcutaneous implantable cardioverter defibrillator with multiple small active can generators and multiple sensing electrodes.

FIG. 6 illustrates a subcutaneous implantable cardioverter defibrillator with multiple shock electrodes and multiple sensing electrodes.

FIG. 7 illustrates a subcutaneous implantable cardioverter defibrillator with multiple shock electrodes, a split-active can generator and multiple sensing electrodes.

### DETAILED DESCRIPTION OF ONE OR MORE EMBODIMENTS

The disclosure is particularly applicable to a multi-vector patient electrode system that may be used with an internal cardioverter defibrillator or a subcutaneous internal cardioverter defibrillator which are used for sensing and terminating Atrial Fibrillation and other non-lethal cardiac arrhythmias in addition to Ventricular Fibrillation and Ventricular Tachycardia, and it is in this context that the disclosure will be described. It will be appreciated, however, that the multi-vector patient electrode system has greater utility since it may be used with any medical device or other system in which it is desirable to be able to deliver an electric or therapeutic pulse via multiple pathways, whether simultaneously or sequentially or with some greater or lesser degree of overlap in the timing of the pulse deliveries.

While the physical alignment within 3 dimensions of the cardiac fibers and myocytes of an actual patient is not knowable in an actual patient at the time of delivering a shock, if multiple vectors are used for/within the same pulse then this will effectively increase the number of cells affected and depolarized and so increase the probability of a successful defibrillation. Thus, a system capable of delivering shocks across multiple vectors increases the probability of successful defibrillation by correcting for the potentially suboptimal vector used in an emergency situation. Furthermore, traditional systems have all used biphasic shocks. The ability to deliver a wide variety of multiphasic shocks, across multiple vectors will introduce significant advantages into clinical use by combining novel form factors (small, always available, and with distributed shock vectors) and novel waveforms to solve the probem of readily available sources of successful defibrillation to treat Ventricular Fibrillation. This approach also provides significant clinical advantages in the form of increased efficaciousness over existing approaches for lower shock energies. Thus, a subcutaneous device composed of multiple shock electrodes which provide two or more shock vectors, rather than the standard single shock vector, would offer the ability to defibrillate effectively with higher probabilities of shock success and at lower energies than single vector systems traditionally can.

A subcutaneous device may be composed of two pulse generators-like components, each placed laterally (left sided and right sided) would be an attractive alternative since it may permit each implanted component to be small, and would provide an attractive vector with high efficiency. Each pulse generator includes an energy reservoir since each pulse generator can generate its pulse using its energy reservoir. A system that distributed the electronic components may permit very small components. Each of these smaller generators may be connected to the multiple shock electrodes. The two or more smaller generators may be then connected to each other electrically by electrical leads once they are implanted into the patient. In addition, the lead connecting the two components could also serve as a lead for sensing and defibrillation. Other embodiments of the device may provide shock vector options via use of multiple coils located sternally and a left-lateral “active can” generator with two distinct and separate electrode surfaces upon its exterior.

The use of multiple energy reservoirs allows for the generation and delivery of multiple pulses to the patient. Alternatively, the multiple energy reservoirs permit each phase of a multiphasic pulse to be separately generated and separately delivered. These can be delivered via one or more different shock vectors if the energy reservoirs are connected to a plurality of electrodes. This disclosure allows for the separate pulses or even the separate phases of a multiphasic pulse to be delivered via different shock vectors (including through completely different combinations of electrodes) in order to enhance the overall percentage of cardiac tissue that is successfully defibrillated or cardioverted and so more effectively terminate the lethal/non-lethal arrhythmia in the patient. This disclosure also allows for the static or dynamic configuration of one-to-many and many-to-one shock vector arrangements as an alternate or additional method of enhancing the overall percentage of cardiac tissue that is successfully defibrillated or cardioverted. This disclosure also allows for the individual pulses or pulse phases to be delivered in a manner that overlaps in the timing to a greater or lesser degree. This approach can also be used for delivering electrical pulses, at any level of energy, in other therapeutic and clinical areas outside of cardiac stimulation in order to cause a specific therapeutic outcome in a patient such as in the fields of neurological stimulation, gastrointestinal stimulation or the stimulation of specific internal organs or nerve systems within a patient's body.

The multi-vector patient electrode system may also include and employ a mix of sensor types, such as ECG sensors and LED optical pulse detectors, in addition to or combined with the therapeutic shock electrodes. This mix means that the internal cardioverter defibrillator's accuracy of the detection of shockable arrhythmias can be significantly improved. The mix of sensor types may further include sensors which can be active in nature, passive in nature, or a combination of the two types.

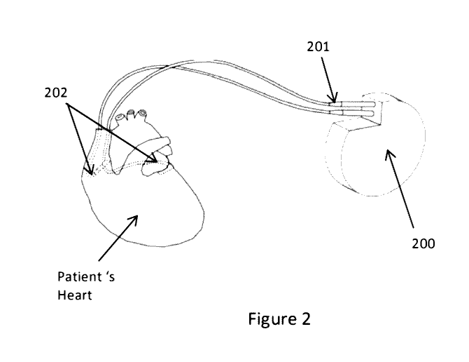

FIG. 2 illustrates a standard ICD (200) with a single shock vector electrode system, used by the majority of current internal cardioverter defibrillators. The transvenous lead(s) (201) link the active generator unit to the shock electrode(s) positioned in the relevant chamber(s) of the heart (202) and the ICD then selects the appropriate lead for generating the shock vector according to the type of arrhythmia detected and the chamber of the heart that needs to be shocked. The single vector shock is then delivered between the active lead and the active generator as appropriate.

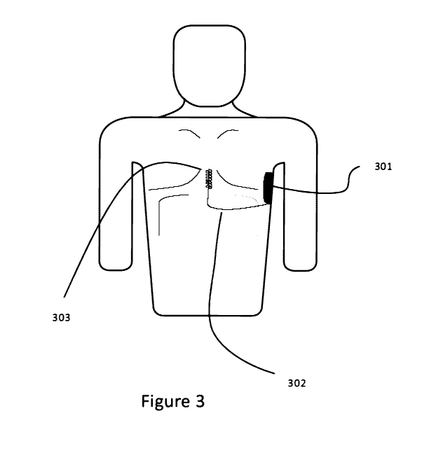

FIG. 3 illustrates a standard S-ICD “active can” generator (301) with a single shock vector electrode (303) connected via a single lead (302). As shown the generator (301) is implanted on the left-lateral side of the patient with the lead (302) tunneled under the skin and the electrode (303) likewise tunneled under the skin and positioned over the sternum. This results in a single shock vector system, between the active can pulse generator and the electrode, which relies upon the exact positioning of both the generator and the electrode at the time of implantation.

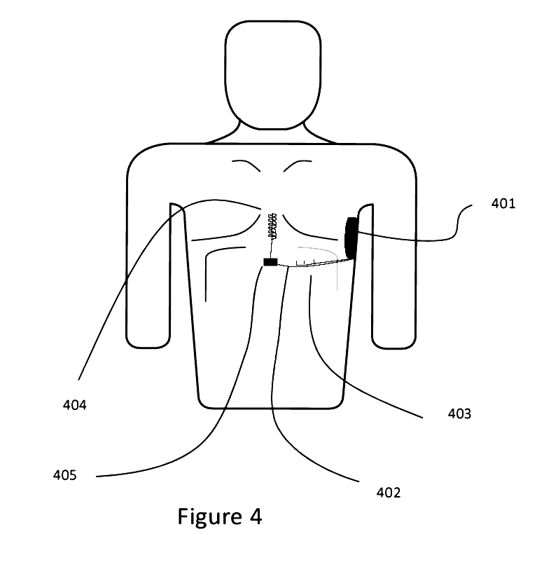

FIG. 4 illustrates a novel S-ICD system making use of a multi-vector electrode system. An active can pulse generator (401) may be positioned in the standard left-lateral position under the skin and connected to a sternal conductive patient electrode (404) also placed under the skin via a lead (402). In addition, the lead (402) can include a plurality of ECG and pulse sensors/electrodes (403) along its length which allows for the sensing of a multi-lead ECG signal, the quality of which is dependent upon the number of sensors/electrodes utilized. The system may also have a small additional housing/junction (405), which is either positioned sternally or ad xiphoid, in between the electrode (404) and the sensing electrodes (403) which may contain an additional shock electrode and/or additional sensors and/or other components according to the exact embodiment required. This addition of a third active electrode in the additional housing/junction (405) enables the use of multiple shock vectors delivered to the patient by the system between the generator (401), the electrode (404) and the additional housing (405).

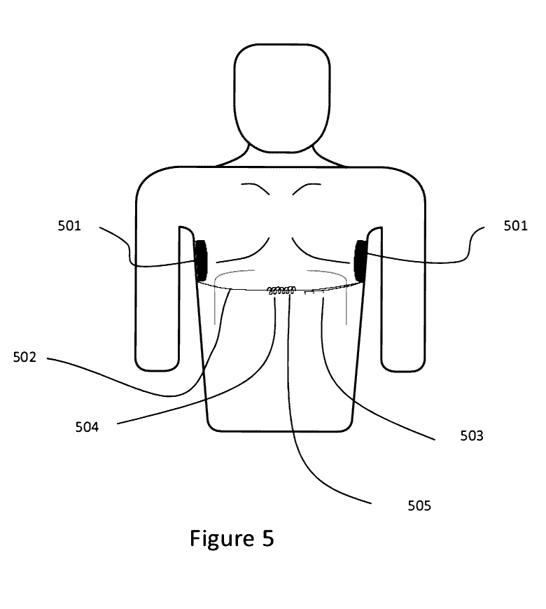

FIG. 5 illustrates a novel S-ICD system 500 that has a multi-vector electrode system and a multi-generator system. In the system, an active can pulse generators (501) may be positioned in the left-lateral and right-lateral positions under the skin. Each active can pulse generator in this embodiment and in the other described embodiments may contain an energy reservoir and circuitry and be capable of generating a pulse or a phase of the pulse so that defibrillation shocks may be delivered to the patient over multiple shock vectors. In some embodiments, the pulse generator may generate a multi-phasic pulse (a pulse with multiple phase signals such as one or more positive phase signals and one or more negative phase signals) and the different phase signals may be delivered to the patient over the multiple shock vectors. The active can pulse generators 501 are connected to each other via a subcutaneous lead (502) also placed under the skin across the torso of the patient. The lead may have one or more shock electrodes (504; 505) along its length. In addition the lead (502) can include a plurality of ECG and pulse sensors/electrodes (503) along its length which allows for the sensing of a multi-lead ECG signal, the quality of which is dependent upon the number of sensors/electrodes utilized. This combination of the two active can pulse generators and the one or more additional shock electrodes enables the delivery of shocks over multiple shock vectors by the system between either of the generators (501), and either of the electrodes (504; 505) or any suitable combination of these.

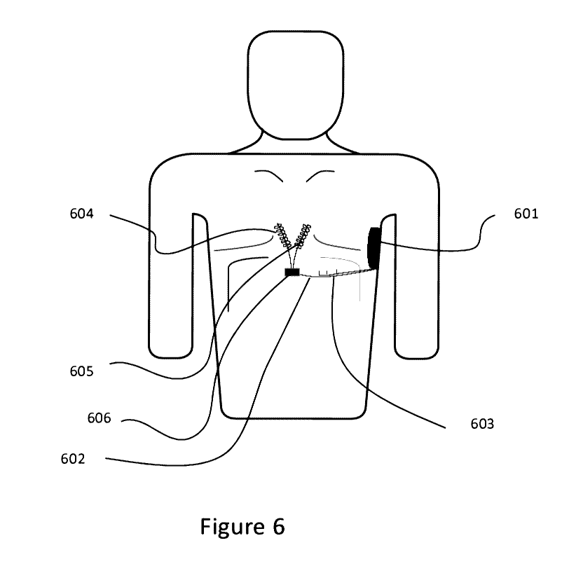

FIG. 6 illustrates a novel S-ICD system 600 that includes a multi-vector electrode system. An active can pulse generator (601) is positioned in the standard left-lateral position under the skin and connected to a sternal or ad xiphoid housing/junction (606) also placed under the skin via a lead (602). In addition, the lead (602) can include a plurality of ECG and pulse sensors/electrodes (603) along its length which allows for the sensing of a multi-lead ECG signal, the quality of which is dependent upon the number of sensors/electrodes utilized. The sternal housing/junction (606) is connected to the two sternal electrodes (604; 605) and may also contain additional components and sensors. This option of multiple active sternal electrodes (604; 605) enables the delivery of shocks over multiple shock vectors by the system between the generator (601), and the electrodes (604; 605).

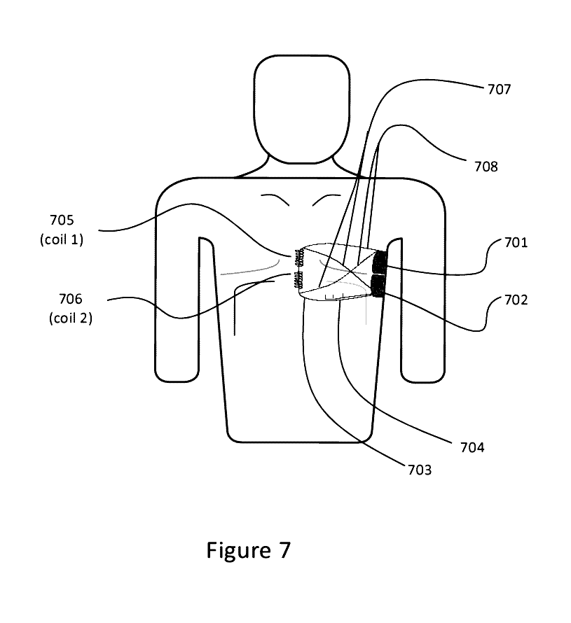

FIG. 7 illustrates a novel S-ICD system that has a multi-vector electrode system. A single pulse generator has an exterior consisting of two separate active can portions (701; 702) and it is positioned in the standard left-lateral position under the skin. The pulse generator is connected to each of the two or more sternal shock electrodes (705; 706) via a subcutaneous lead (703) also placed under the skin of the patient. In addition the lead (703) can include a plurality of ECG and pulse sensors/electrodes (704) along its length which allows for the sensing of a multi-lead ECG signal, the quality of which is dependent upon the number of sensors/electrodes utilized. This combination of the two active can portions (701; 702) of the pulse generator and the one or more additional shock electrodes (705; 706) enables the delivery of shocks over multiple shock vectors by the system between either of the generator portions (701; 702), and either of the electrodes (705; 706) or any suitable combination of these. Examples of these potential shock vectors are shown (707; 708).

The multi-vector patient electrode system may be placed into a body of a patient and may be used, for example, to deliver one or more therapeutic pulse(s) to the patient for defibrillation or cardioversion. The multi-vector patient electrode system may also be used to deliver other types of treatments of varying energies and durations to the patient, such as neurological stimulation, gastrointestinal stimulation or the stimulation of specific internal organs or nerve systems within a patient's body. The multi-vector patient electrode system may also be used to sense a characteristic of the patient, such as a heartbeat or pulse and the like. The multi-vector patient electrode system may also be used to both sense a characteristic of the patient and deliver a treatment to the patient when the embodiment of the multi-vector patient electrode system makes use of both sensors and electrodes.

The multi-vector patient electrode system may be placed into the body of the patient at various locations, such as the torso, abdomen, limbs and/or head of the patient. In some implementations, multiple multi-vector patient electrode system may be used and each multi-vector patient electrode system may be placed in one or more locations in the body of the patient.

In the various example embodiments described above, the pulse delivered to the patient using the multi-vector patient electrode system may be multiphasic pulse that may have one or more different phases of the pulse. In some embodiments, each phase of the multiphasic pulse may be delivered via its own shock vector using the multi-vector patient electrode system. In some embodiments, the one or more phases of the multiphasic pulse may be delivered via a shock vector previously used within the same pulse. In some embodiments, each phase of the multiphasic pulse may be delivered within its own unique segment of the overall pulse timing sequence. In some embodiments, the one or more phases of the multiphasic pulse may be delivered within a time segment that overlaps to a greater or lesser degree with one or more of the other timing segments in the overall pulse sequence.

In the various example embodiments described above, the one or more conductive patient electrodes may be each connected to separate individual electrical lead. In other embodiments, a plurality of the more than one conductive patient electrodes may be connected to the same electrical lead.

The multi-vector patient electrode system may be placed under the skin/surface of the body of the patient. For example, the multi-vector patient electrode system may be placed in the torso of the patient, the abdomen of the patient, a limb of the patient and the head of the patient.

In the various example embodiments described above, the one or more conductive electrodes may have one or more of a variety of shapes. In other embodiments, the one or more conductive electrodes may have one or more different sizes. The one or more conductive electrodes may be also anchored in place within the patient.

In some embodiments, the system may include one or more patient sensors. The one or more patient sensors may actively or passively sense one or more of a variety of biometric readings from the patient. The biometric readings from the patient may include an ECG signal. In some embodiments, the one or more sensors are arranged separately from the one or more conductive electrodes.

In the various embodiments described above, the pulses (or phases of the pulse) may be delivered to the patient using one or more shock vectors. Those shock vectors may be selected either statically or dynamically by a medical professional, the manufacturer of the device or an algorithm within the programming of the pulse generator in the device. In the above delivery of the one or more shock vectors, the shock vector may be a path from one-electrode-to-one-electrode, a path from one-electrode-to-many-electrodes, a path from many-electrodes-to-one-electrode and a path from many-electrodes-to-many-electrodes.

The multi-vector patient electrode system may be used to deliver a multi-vector pulse waveform to a patient. In the method, one or more multi-vector patient electrode systems are installed within a patient and the one or more multi-vector patient electrode systems generate a multi-vector pulse waveform to the electrical leads and conductive electrodes of the multi-vector patient electrode systems. The multi-vector pulse waveform is delivered to the patient via the one or more conductive electrodes. The multi-vector pulse waveform may be delivered through the one or more conductive electrodes via one or more specific vectors and these vectors are selected either statically or dynamically by one or more of a medical professional, the manufacturer or an algorithm within the programming of the pulse generator. The one or more vectors selected are of at least a one-electrode-to-one-electrode, a one-electrode-to-many-electrode, a many-electrode-to-one-electrode, and a many-electrode-to-many-electrode nature. In the method, the one or more phases of a multiphasic pulse waveform are each routed via the same selected vector, the one or more phases of a multiphasic pulse waveform are each routed via different selected vectors and/or the one or more phases of a multiphasic pulse waveform are each routed via a combination of the same selected vector and different selected vectors.

While the foregoing has been with reference to a particular embodiment of the invention, it will be appreciated by those skilled in the art that changes in this embodiment may be made without departing from the principles and spirit of the disclosure, the scope of which is defined by the appended claims.

1. An implantable multi-vector patient electrode system, comprising:
one or more conductive electrodes;
one or more electrical leads connected to the one or more conductive electrodes;
one or more pulse generators, electrically connected to the one or more electrical leads, that each generate a pulse to be delivered to a patient.

2. The implantable multi-vector patient electrode system of claim 1, wherein the pulse from the one or more pulse generators is delivered to the patient over multiple shock vectors using the one or more electrical leads and the one or more conductive electrodes.

3. The implantable multi-vector patient electrode system of claim 2, wherein the pulse delivered is a multiphasic pulse.

4. The implantable multi-vector patient electrode system of claim 3, wherein each phase of the multiphasic pulse is delivered via its own shock vector.

5. The implantable multi-vector patient electrode system of claim 3, wherein one or more phases of the multiphasic pulse is delivered via a shock vector previously used within the same pulse.

6. The implantable multi-vector patient electrode system of claim 3, wherein each phase of the multiphasic pulse is delivered within its own unique segment of the overall pulse timing sequence.

7. The implantable multi-vector patient electrode system of claim 3, wherein one or more phases of the multiphasic pulse is delivered within a time segment that overlaps to a greater or lesser degree with one or more of the other timing segments in the overall pulse sequence.

8. The implantable multi-vector patient electrode system of claim 1, wherein the one or more conductive electrodes are each connected to a separate electrical lead.

9. The implantable multi-vector patient electrode system of claim 1, wherein the one of more conductive electrodes are connected to the same electrical lead.

10. The implantable multi-vector patient electrode system of claim 1, wherein the multi-vector patient electrode system is placed under the skin/surface of the body of the patient.

11. The implantable multi-vector patient electrode system of claim 10, wherein the multi-vector patient electrode system is placed in one or more of the torso of the patient, the abdomen of the patient, a limb of the patient and the head of the patient.

12. The implantable multi-vector patient electrode system of claim 1, wherein the one or more conductive electrodes have one or more of a variety of shapes.

13. The implantable multi-vector patient electrode system of claim 1, wherein the one or more conductive electrodes have one or more different sizes.

14. The implantable multi-vector patient electrode system of claim 1, wherein the one or more conductive electrodes are anchored in place within the patient.

15. The implantable multi-vector patient electrode system of claim 1 further comprising one or more sensors.

16. The implantable multi-vector patient electrode system of claim 15, wherein the one or more sensors actively or passively sense one or more of a variety of biometric readings from the patient.

17. The implantable multi-vector patient electrode system of claim 16, wherein the one or more biometric readings from the patient is an ECG signal.

18. The implantable multi-vector patient electrode system of claim 15, wherein the one or more sensors are arranged separately from the one or more conductive electrodes.

19. The implantable multi-vector patient electrode system of claim 1, wherein the one or more vectors selectable are of at least a one-electrode-to-one-electrode, a one-electrode-to-many-electrode, a many-electrode-to-one-electrode, and a many-electrode-to-many-electrode nature.

20. A method for installing a multi-vector patient electrode system into a patient, the method comprising:
providing one or more multi-vector patient electrode systems wherein the one or more multi-vector patient electrode systems are arranged in a configuration to provide optimal positioning within a patient for the desired multi-vector shock delivery; and
placing the multi-vector patient electrode system within the body of a patient.

21. The method of claim 20, wherein placing the multi-vector patient electrode system further comprises placing one or more multi-vector patient electrode systems at a location within the body of the patient.

22. The method of claim 21, wherein the location within the body of the patient is at least one of a torso of the patient, the abdomen of the patient, a limb of the patient and a head of the patient.

23. The method of claim 20 further comprising delivering, using the multi-vector patient electrode system, a treatment to the patient.

24. A method for delivering a multi-vector pulse waveform to a patient, the method comprising:
installing one or more multi-vector patient electrode systems within a patient;
generating, using the multi-vector patient electrode systems, a multi-vector pulse waveform to electrical leads and one or more conductive electrodes of the multi-vector patient electrode systems; and
delivering the multi-vector pulse waveform to the patient via the one or more conductive electrodes.

25. The method of claim 24, wherein the multi-vector pulse waveform is delivered through the one or more conductive electrodes via one or more specific vectors and these vectors are selected either statically or dynamically by one or more of a medical professional, the manufacturer or an algorithm within the programming of the pulse generator.

26. The method of claim 25, wherein the one or more vectors selected are of at least a one-electrode-to-one-electrode, a one-electrode-to-many-electrode, a many-electrode-to-one-electrode, and a many-electrode-to-many-electrode nature.

27. The method of claim 26, wherein the one or more phases of a multiphasic pulse waveform are each routed via the same selected vector.

28. The method of claim 26, wherein the one or more phases of a multiphasic pulse waveform are each routed via different selected vectors.

29. The method of claim 26, wherein the one or more phases of a multiphasic pulse waveform are each routed via a combination of the same selected vector and different selected vectors.
